(19) United States
(12) Patent Application Publication
(10) Pub. No.: US 2024/0181419 A1
(43) Pub. Date: Jun. 6, 2024

(54) APPARATUS AND METHOD FOR REMOVING PROTEINS TAKEN UP IN A CARRIER LIQUID

(71) Applicant: LUXEMBOURG INSTITUTE OF SCIENCE AND TECHNOLOGY, Esch-sur-Alzette (LU)

(72) Inventors: Marcelo FERNANDEZ LAHORE, Bremen (DE); Lisa-Marie HERLEVI, Bremen (DE)

(73) Assignee: LUXEMBOURG INSTITUTE OF SCIENCE AND TECHNOLOGY, Esch-sur-Alzette (LU)

(21) Appl. No.: 18/553,401

(22) PCT Filed: Apr. 1, 2022
(86) PCT No.: PCT/EP2022/058755

§ 371 Date: Sep. 29, 2023

(30) Foreign Application Priority Data
Apr. 1, 2021 (DE) 10 2021 108 406.7

(51) Int. Cl. B01J 8/26; B01D 15/18; B01D 15/20; B01D 15/42; B01J 8/34

(52) U.S. Cl. CPC B01J 8/26; B01D 15/1807; B01D 15/203; B01D 15/424; B01J 8/34

(57) ABSTRACT

An apparatus for removing proteins taken up in a carrier liquid, which includes a granular adsorbent for proteins. Also, a method for removing proteins taken up in a carrier liquid, with a corresponding apparatus. Further, the use of a corresponding apparatus, for extracting proteins from a liquid and more particularly from an unclarified feedstock.

### FIELD

The invention relates to an apparatus for removing proteins taken up in a carrier liquid. The invention further relates to a method for removing proteins taken up in a carrier liquid. The invention also relates to the use of a corresponding apparatus, for extracting proteins from a liquid and more particularly from an unclarified feedstock.

### BACKGROUND

Known apparatus and also methods for removing proteins taken up in a carrier liquid have several drawbacks: there is a lack of continuous adsorption processes in the downstream processing of bioproducts, thus generating capacity constraints in industrial manufacturing. Additionally, the first steps of downstream processing, centrifugation and membrane filtration, result in long processing time and significant losses in yield. Moreover, in continuous systems, a corresponding adsorbent cannot be brought for sufficient time and/or sufficient intensity into contact with a corresponding carrier liquid which contains the proteins. Continuous systems based on fluidized bed technology generally suffer from low resin binding capacities and/or a limited flowrate at the adsorption stage, and/or inability to increase the target protein concentration at the elution stage.

### SUMMARY

The aim of the present invention is to improve on the prior art.

The aim is achieved with the first, second and third objects of the present invention as described below.

A first object of the present invention is an apparatus for removing proteins taken up in a carrier liquid, said apparatus having a bottom side disposed in gravitational direction and a top side disposed opposite to the gravitational direction, where the carrier liquid can be brought, and preferably is brought, into contact with a granular adsorbent for the proteins, which is located in a vertically disposed first subreactor, by means of a flow of the carrier liquid around the adsorbent, so that the granular adsorbent takes up and temporarily stores proteins, and the granular adsorbent, saturated partially or completely with proteins, can be transferred, and preferably is transferred, into a second subreactor by means of a first pumping device or a first valve disposed on the top side and between the first subreactor and the second subreactor, in particular so that deposition of the proteins from the granular adsorbent into a washing liquid is enabled in the second subreactor, characterized in that the carrier liquid can be introduced, and preferably is introduced, into the first subreactor by means of a first feed at a subregion, facing the bottom side, of the first subreactor, so that the granular adsorbent and the carrier liquid can flow, and preferably flow, through the first subreactor in co-current opposite to the gravitation, from the bottom side to the top side, by the carrier liquid entraining some or all of the granular adsorbent with it.

Because the carrier fluid can be brought or introduced, and preferably is brought or introduced, into the first subreactor on the bottom side of the first subreactor, the granular adsorbent is passed in co-current with the carrier liquid through the first subreactor. The granular adsorbent here is carried partially by the carrier liquid and partially by collision with other granular adsorbent particles. All in all, therefore, the contact time between granular adsorbent and the carrier liquid is increased, and operation with high throughput is possible, so that a greater amount of the proteins passes into the adsorbent. The adsorbent is then transferred into a second subreactor and, correspondingly, proteins taken up by the adsorbent (unbound proteins) can then be washed off from the adsorbent by means of a washing liquid. Additionally, the apparatus of the invention can be considered as an apparatus for adsorbing proteins taken up in a carrier liquid. Indeed, thanks to the apparatus of the present invention, and more particularly the first and second subreactors, the adsorbent adsorbs the proteins present in the carrier liquid within the first subreactor; and then the adsorbent loaded or saturated with the proteins is washed within the second subreactor.

In the first subreactor, the carrier liquid and the granular adsorbent are continuously added from the bottom part of the first subreactor. Simultaneously, the granular adsorbent loaded with the proteins is removed from the top of the first subreactor and moved to the next stage in the second subreactor.

The carrier liquid is preferably an unclarified feedstock.

Advantageous embodiments of the invention are detailed below.

In one advantageous embodiment here, the first subreactor and/or the second subreactor are or is a fluidized bed reactor, and more advantageously the first subreactor is a fluidized bed reactor. This enables additionally increasing the contact time and contact intensity between the granular adsorbent and the carrier liquid and/or application of unclarified feed.

The first subreactor or fluidize bed reactor is preferably an adsorption column.

The adsorption column has preferably an aspect ratio (which corresponds to the height in cm of the column divided by the diameter in cm of the column) ranging from 15 to 60, and more preferably from 30 to 50.

In one advantageous embodiment, the washing liquid can be introduced, and preferably is introduced, into the second subreactor by means of a second feed on the bottom side of the second subreactor, so that the saturated granular adsorbent and the washing liquid can flow, and preferably flow, through the second subreactor in counter-current, by the washing liquid flowing from the bottom side to the top side and the granular adsorbent flowing with the gravitation from the top side to the bottom side, and in particular a washed granular adsorbent freed wholly or partially from the proteins can be transferred, and preferably is transferred, into the first subreactor on the bottom side of the second subreactor by means of a second pumping device or a second valve, so that repeated utilization of the granular adsorbent is enabled.

Indeed, the contact time is additionally increased further by the passing of the washing liquid and the granular adsorbent in counter-current, since the adsorbent drops or trickles along the gravitation through the liquid medium, namely the washing liquid, which flows opposite to the gravitation. Indeed, the granular adsorbent is carried by gravitational force and slowed down thanks to the washing liquid at counter-current.

The second subreactor is preferably a solid-liquid contactor system, and more preferably a solid-liquid counter-current contactor system.

The granular adsorbent freed wholly or partially from the proteins is preferably transferred from the bottom side of the second subreactor to the first subreactor for reuse by means of a second pumping device.

In one advantageous embodiment, the second subreactor has separate reaction spaces for washing, for eluting and/or for equilibrating, where in particular the respective reaction space is connected to the respectively other reaction space or respectively other reaction spaces by means of a respective transition cross section narrowed (or reduced) relative to a cross section of the respective reaction space.

In the present invention, equilibrating can also mean equilibrating and/or regenerating.

The separate reaction spaces for washing, for eluting and for equilibrating can also be called hereafter washing chamber, eluting chamber and equilibrating chamber respectively.

The separate reaction spaces for washing, for eluting and/or for equilibrating are preferably disposed in series (preferably in the order as mentioned, from the top of the second subreactor to the bottom of the second reactor).

Wash, elution and equilibration stages are respectively performed in the washing chamber, eluting chamber and equilibrating chamber. They are preferably operated in counter-current mode by adding the saturated adsorbent at the top of the second subreactor, resulting in a downward flow of the adsorbent particles.

During the elution and equilibration stages, proteins are released from the adsorbent and the adsorbent free of proteins is equilibrated and/or regenerated.

The narrowed transition cross sections here enable controlling the transition of the granular adsorbent between the corresponding reaction spaces.

Preferably, each narrowed transition cross section of the apparatus comprises a solenoid pinch valve, notably which can allow such control.

In one advantageous embodiment, the second subreactor and/or the respective reaction space have or has a (respective) baffle element or a plurality of (respective) baffle elements, where the respective baffle element limits the respective cross section of the second subreactor and/or of the respective reaction space, so that a contact frequency and/or a residence time of the granular adsorbent in the second subreactor and/or in the respective reaction space is increased, in particular by a decelerating and/or fluidizing of a flow of the washing liquid, an eluting liquid, and/or an equilibrating liquid.

Indeed, where a baffle element or a plurality of baffle elements are disposed in the corresponding second subreactor and/or in a first reaction space and/or in the separate reaction spaces, the granular adsorbent may additionally be hindered in its movement and, moreover, the contact time between the granular adsorbent and the washing liquid, the eluting liquid, and/or the equilibrating liquid is additionally increased. In this way a residence time of the adsorbent in the second subreactor and/or in the respective reaction space(s) is increased, in particular by the decelerating and/or fluidizing of a flow of the washing liquid, an eluting liquid, and/or an equilibrating liquid.

The baffle elements are preferably alternatively disposed in the second subreactor in an overlapping manner and without any contact between each other. In other words, baffle elements are preferably alternatively disposed on both sides of the second subreactor, where each baffle element overlaps another baffle element without any contact between each other.

This specific modified inside structure of the second subreactor controls the sedimentation velocity of the adsorbent particles, leading to increased residence time and packing of adsorbent particles.

A (respective) baffle element of this kind is, for example, an (respective) impact face, which is disposed in particular at an angle, for example, between 25° and 60° (boundaries included) or between 60° and 85° (boundaries included), and more preferably between 60° and 85° (boundaries included), obliquely to a flow direction of the washing liquid (or the eluting liquid or the equilibrating liquid). A baffle element of this kind is configured, for example, as an impact face, more particularly as a plate or a baffle plate.

A corresponding, preferably small, passage may serve for passage of the granular adsorbent.

In an advantageous embodiment, the (respective) baffle element and/or the (respective) impact face have or has a passage on a side facing the bottom side, so that the adsorbent can be conveyed, and preferably is conveyed, from the top side to the bottom side by means of gravitation

In a particularly advantageous embodiment, the baffle elements are present in the eluting and equilibrating chambers.

The baffle elements can be present in the washing, eluting and equilibrating chambers, even if it is not the preferred embodiment.

The granular adsorbent is preferably brought between the first subreactor and the second subreactor in each case with a pumping device or a valve, said pumping device being configured in particular as a peristaltic pumping device to the adsorbent. Other pumping devices are also possible such as a diaphragm pump or a screw transporter.

In one preferred embodiment, the adsorbent is conveyed from the first subreactor to the second subreactor for the wash stage by means of a first valve.

In an advantageous embodiment, the first pumping device and/or the second pumping device have or has a peristaltic pumping device for transferring the adsorbent from the first subreactor into the second subreactor and/or from the second subreactor into the first subreactor.

A peristaltic pumping device of this kind or a valve is able to convey a correspondingly granular adsorbent within the flow without moving parts, and at the same time retains a large part of the carrier liquid and/or washing liquid.

In one embodiment, the apparatus further comprises a collecting plate in the first subreactor, for example to improve the transport of the granular adsorbent from the top side of the first subreactor to the top side of the second subreactor.

In one embodiment, the apparatus further comprises a screen disposed respectively on the bottom side and also on a top side of the first subreactor, so that the carrier liquid may flow through the first subreactor in a flow direction. The screen is in each case preferably dimensioned such that the granular adsorbent is retained and only the carrier liquid is able to pass through.

Similarly to this, the apparatus can be provided on the top side and on the bottom side of the second subreactor with a screen, so that a washing liquid flowing through the second subreactor may be able to flow through along a flow direction, with the granular adsorbent remaining in the apparatus.

The granular adsorbent here is formed, for example, of granular grains between 0.1 mm and 0.5 mm, and preferably between 0.2 mm and 0.5 mm.

In one preferred embodiment, the granular adsorbent is in the form of monodisperse granular grains. In the present invention the term “monodisperse” means that the granular grains have a polydispersity index (PDI) lower than 1.1 and greater than 1.

In particular the granular adsorbent has a density of at least 1.1 g/ml, preferably ranging from 1.1 to 4.4 g/ml, more preferably from 1.1 to 1.4 g/ml, and even more preferably from 1.1 to 1.2 g/ml.

In particular, the granular adsorbent is an ion-exchanger and preferably a cation exchanger.

The granular adsorbent can be of the hydrophobic interaction, mixed-mode or affinity type-depending on the properties of the protein to be removed or recovered.

In one preferred embodiment, the granular adsorbent has an open porosity.

In an advantageous embodiment, the granular adsorbent displays pores with a size ranging from 15 to 1500 Angstroms, and more advantageously from 300 to 1000 Angstroms.

In the present invention, the pore size is determined by commonly known methods, such as gas sorption method, mercury intrusion method, or inverse size-exclusion chromatography.

The granular adsorbent can comprise or be composed of polymer matrix and an entrapped densifier; or a modified dense porous inorganic material.

The polymer matrix can be selected from (cross-linked) agarose, dextran, cellulose, vinyl alcohol polymers and copolymers, methacrylate polymers and copolymers, acrylate polymers and copolymers.

The entrapped densifier can be selected from a metal-based material, silica, glass, quartz, and quartz sand.

As examples of metal-based materials, stainless steel, ZrO₂, tungsten carbide, and Nd—Fe—B alloy are preferred.

The modified dense porous inorganic material can be selected from glass, zirconia, ceramic hydroxyapatite, and fluor hydroxyapatite.

In one preferred embodiment, the granular adsorbent comprises:
agarose and quartz,
agarose and glass,
agarose and a metal-based material as defined above, and preferably agarose and tungsten carbide, or
dextran and silica.

A second object of the present invention is a method for removing proteins taken up in a carrier liquid, with an apparatus as defined in the first object of the present invention, with the steps of:
introducing the carrier liquid and the granular adsorbent on the bottom side of the first subreactor by means of the first feed or introducing the carrier liquid on the bottom side of the first subreactor by means of the first feed, said first subreactor being pre-loaded with the granular adsorbent (first stage),
bringing the carrier liquid from the bottom side of the first subreactor to the top side of the first subreactor, so that the granular adsorbent is brought in co-current with the carrier liquid and the granular adsorbent is held at least partially in suspension, so that a contact time of the granular adsorbent with the carrier liquid is increased (second stage),
transferring the granular adsorbent (loaded with the proteins) from the first subreactor into the second subreactor by means of the first pumping device or the first valve, in particular so that deposition of the proteins from the granular adsorbent into the washing liquid is enabled (third stage),
so that the proteins are removed from the carrier liquid.

Thanks to the apparatus of the invention, the method is a continuous method.

The method here is performed with an apparatus according to one or more of the above-described embodiments in the first object of the present invention.

Here in particular (in the first and second stages), the carrier liquid and the adsorbent are introduced on the bottom side of the first subreactor, so that the carrier liquid and the granular adsorbent can be conveyed, and preferably are conveyed, in co-current. This is accomplished by means of the carrier liquid being brought from the bottom side of the first subreactor to the top side of the first subreactor.

Where the granular adsorbent is saturated wholly or partially with corresponding proteins, the adsorbent is transferred from the first subreactor into the second subreactor by means of the first pumping device or the first valve (third stage). In this way the advantages of the above-described apparatus can be exploited as effectively as possible with this method.

Following the transfer into the second subreactor, the granular adsorbent may also be passed in counter-current with the washing liquid (fourth stage or wash stage). This takes place as described above.

In an advantageous embodiment, the granular adsorbent, after the transferring into the second subreactor, is passed in counter-current with the washing liquid, so that the granular adsorbent flows with the gravitation and the washing liquid flows opposite to the gravitation (fourth stage).

The washed granular absorbent is free of impurities after the fourth stage.

The washed granular absorbent can then be subjected to an eluting stage (fifth stage or elution stage) so as to remove the proteins from the washed absorbent.

In this elution stage, the washed granular absorbent is put into contact with an eluting liquid.

More particularly, the washed granular adsorbent is passed in counter-current with the eluting liquid, so that the washed granular adsorbent flows with the gravitation and the eluting liquid flows opposite to the gravitation.

The method can further comprise an equilibration and/or regeneration stage of the granular adsorbent (sixth stage). The obtained adsorbent can then be reused with a new carrier liquid.

In this stage, the granular absorbent (free of proteins) is put into contact with an equilibrating and/or regenerating liquid.

More particularly, the granular adsorbent is passed in counter-current with the equilibrating and/or regenerating liquid, so that the granular adsorbent flows with the gravitation and the equilibrating and/or regenerating liquid flows opposite to the gravitation.

A third object of the present invention is the use of an apparatus as defined in the first object of the present invention for extracting proteins from a liquid, preferably an unclarified feedstock.

### BRIEF DESCRIPTION OF THE DRAWINGS

The invention is elucidated in more detail below using exemplary embodiments. In this connection

FIG. 1 shows a schematic representation of an apparatus for removing proteins taken up in a carrier liquid, in a side elevation, according to one embodiment of the invention.

FIG. 2 shows a schematic representation of an apparatus for removing proteins taken up in a carrier liquid, in a side elevation, according to another embodiment of the invention.

### DETAILED DESCRIPTION

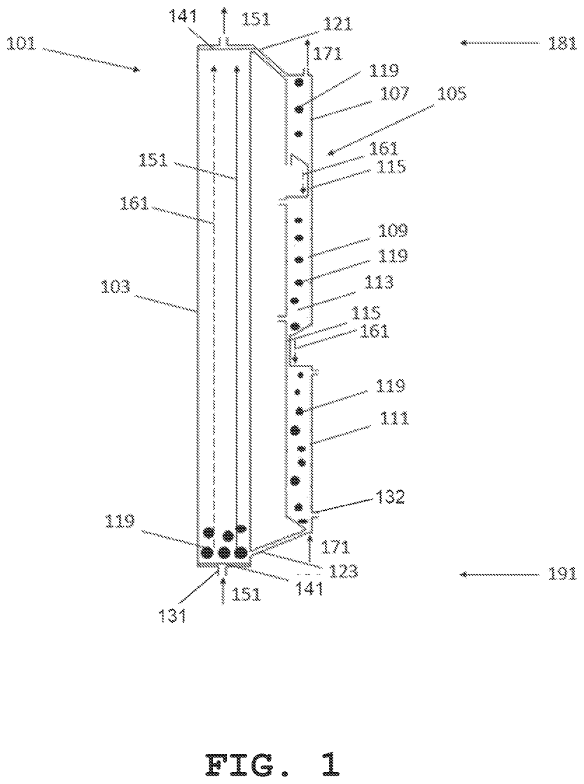

In FIG. 1, an apparatus 101 has a first subreactor 103 and also a second subreactor 105. The second subreactor 105 is formed of a washing chamber 107 (also called separate reaction space for washing), an eluting chamber 109 (also called separate reaction space for eluting) and an equilibrating chamber 111 (also called separate reaction space for equilibrating), which are disposed in series. The corresponding chambers 107, 109 and also 111 are connected to one another by means of transitions (also called transition cross sections) 115, with each transition or transition cross section 115 having a reduced (or narrowed) cross section relative to the respective chamber 107, 109 or 111.

The first subreactor 103 and also the second subreactor 105 are preferably connected on a top side 181 to a pumping device such as a peristaltic pump or a valve 121. By means of the peristaltic pump or the valve 121, the granular adsorbent 119 may be conveyed from the top side 181 of the first subreactor 103 to the top side 181 of the second subreactor 105.

The second subreactor 105 is preferably connected by means of a pumping device such as a peristaltic pump or a valve 123 to the first subreactor 103 on a bottom side 191. By means of the peristaltic pump or the valve 123, corresponding granular adsorbent 119 may be conveyed from the bottom side 191 of the second subreactor to the bottom side 191 of the first subreactor 103.

A carrier liquid (not shown) may flow through the first subreactor 103 in a flow direction 151. For this purpose, a screen 141 can be disposed respectively on the bottom side 191 and also on a top side 181 of the first subreactor 103. The screen 141 is in each case preferably dimensioned such that the granular adsorbent 119 is retained and only the carrier liquid is able to pass through.

Similarly to this, the second subreactor 105 is preferably provided on the top side 181 and on the bottom side 191 with a screen 142 (not shown), so that a washing liquid flowing through the second subreactor 105 may be able to flow through along a flow direction 171, with the granular adsorbent 119 remaining in the apparatus 101.

A corresponding carrier liquid (not shown) provided with proteins flows along the flow direction 151 through the first subreactor 103 on the bottom side 191 to the top side 181. The granular adsorbent 119 here is partially held in suspension and therefore, viewed relatively, flows more slowly to the top side 181 and, in so doing, takes up proteins from the carrier liquid. The granular adsorbent 119 enriched with proteins is then brought into the second subreactor 105 on the top side 181 by means of the pumping device such as peristaltic pump or a valve 121. Here the granular adsorbent 119 trickles opposite to the flow direction 171 of the washing liquid slowly through corresponding chambers 107, 109 and also 111 of the second subreactor 105. The granular adsorbent 119 here flows along the flow direction 161.

More particularly, in the washing chamber 107, the granular adsorbent loaded with proteins is washed with the washing liquid, notably so as to remove impurities, then it is eluate with an eluting liquid so as to release the proteins from the granulate adsorbent in the eluting chamber 109, and then the granulate adsorbent free of proteins is regenerated with an equilibrating and/or regenerating liquid in the equilibrating chamber 111.

The eluate provided with the proteins is then passed along the flow direction 171 from the second subreactor and may be put to further use.

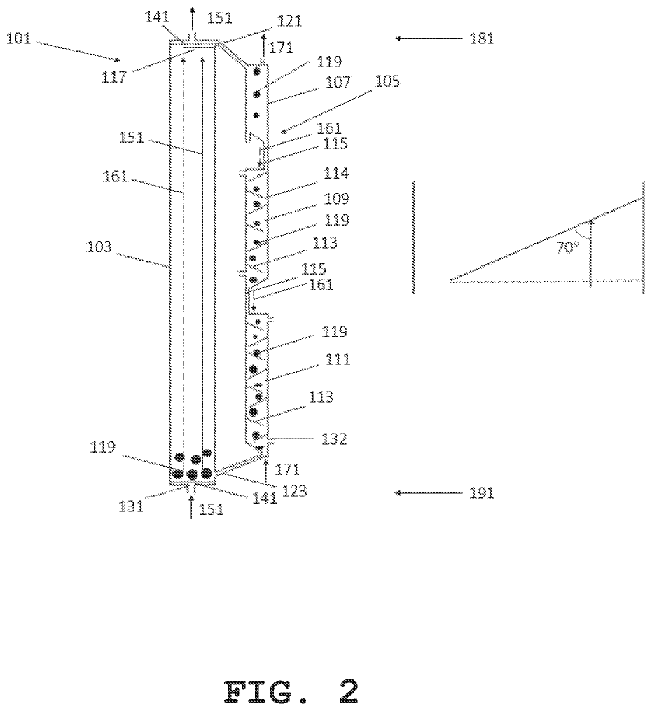

In FIG. 2, the apparatus is identical to the one described above except that said apparatus further comprises a collecting plate 117 and baffles elements 113 such as baffle plates. By means of the peristaltic pump or the valve 121, the granular adsorbent 119 located on said collecting plate 117, may be conveyed from the top side 181 of the first subreactor 103 to the top side 181 of the second subreactor 105. The baffles elements 113 such as baffle plates, are disposed within the eluting chamber 109, and the equilibrating chamber 111, at an angle of about 70° to a traversing flow direction (i.e. obliquely to a flow direction (171) of the washing liquid).

In each case the baffle plates 113 leave small passages 114 to the walls of the respective chambers 107, 109 and 111, through which granular adsorbent 119 is able to trickle.

Here the granular adsorbent 119 is guided over the baffle plates 113 and therefore trickles opposite to the flow direction 171 of the washing liquid slowly through corresponding chambers 107, 109 and also 111 of the second subreactor 105. The granular adsorbent 119 here flows along the flow direction 161.

By means of the baffle plates 113, therefore, the residence of the granular adsorbent 119 in the second subreactor 105 is prolonged, and so proteins adsorbed correspondingly in the granular adsorbent are eluted from this adsorbent by the eluting liquid.

### Example of Method According to the Invention

The carrier liquid which corresponds to a 2 g/l lysozyme feedstock and the granular adsorbent which corresponds to a commercial cationic adsorbent comprising polymethacrylate, Relisorb SP405/EB (Resindion, Italy), are introduced on the bottom side 191 of the first subreactor 103 by means of the first feed 131. The volumetric flowrate of the carrier liquid is of 0.93 L/h (corresponding approximately to a flowrate of 550 cm/h).

The first subreactor 103 is an adsorption column with a column diameter of 1.6 cm at an operational height of 30 cm. When the height of the column is of 60 cm, the volumetric flowrate can be up to 1.3 L/h.

More particularly, the adsorption column 103 is first filled with the adsorbent up to half of the height of the column and then the adsorption column is fed at the bottom side 191 with an equilibrating liquid, which corresponds to a 20 mM sodium phosphate buffer having a pH of 7, with a volumetric flowrate of 0.75 L/h or a flowrate of 400 cm/h until it reaches the top side 181 of the column. Then, the adsorption column 103 is fed at the first feed 131 with the 2 g/l lysozyme feedstock at the bottom side 191 at a volumetric flowrate of 0.93 L/h or a flowrate of 550 cm/h.

The 2 g/l lysozyme feedstock goes from the bottom side 191 of the adsorption column 103 to the top side 181 of the adsorption column 103, so that the granular adsorbent is brought in co-current with the 2 g/l lysozyme feedstock.

Then, the granular adsorbent (loaded with the proteins from the 2 g/l lysozyme feedstock) is transferred from the adsorption column 103 into a second subreactor 105 by means of a valve 121.

The second subreactor 105 comprises a washing chamber 107, an eluting chamber 109 and an equilibrating and/regenerating chamber 111, where the eluting chamber 109 and the equilibrating and/regenerating chamber 111 are manufactured with baffles at 85, 80 or 75 degree angles to assess the elution performance with different retention times of the granulate adsorbent.

A washing liquid which corresponds to a 20 mM sodium phosphate buffer having a pH of 7 is fed at the second feed 132 at the bottom side 191 and flow through the second subreactor 105 in counter-current from the bottom side 191 to the top side 181 at a flowrate of 600 cm/h. Simultaneously, the loaded granular adsorbent flows with the gravitation from the top side 181 to the bottom side 191. At the end of the wash stage (fourth stage), the granular adsorbent is free of impurities.

Then, an eluting liquid which corresponds to a 1 M NaCl in 20 mM sodium phosphate buffer having a pH of 7 is fed at the second feed 132 at the bottom side 191 and flow through the second subreactor 105 in counter-current from the bottom side 191 to the top side 181 at a flowrate of 100 cm/h. Simultaneously, the loaded and washed granular adsorbent flows with the gravitation from the top side 181 to the bottom side 191. At the end of the elution stage (fifth stage), the granular adsorbent is free of proteins and the proteins are in the eluting liquid (eluate).

Then, an equilibrating and/or regenerating liquid which corresponds to a 1 M NaCl in 20 mM sodium phosphate buffer having a pH of 7 (re-equilibrating) or 0.1 M NaOH (regenerating) is fed at the second feed 132 at the bottom side 191 and flow through the second subreactor 105 in counter-current from the bottom side 191 to the top side 181 at a flowrate of 100 cm/h. Simultaneously, the granular adsorbent free of proteins flows with the gravitation from the top side 181 to the bottom side 191. At the end of the equilibration and/or regeneration stage (sixth stage), the granular adsorbent is ready for reuse.

The highest protein concentration in the eluting liquid was achieved with the 85 degree angle column and with all of the columns increased protein concentration was achieved compared to a conventional apparatus.

1-11. (canceled)

12. An apparatus for removing proteins taken up in a carrier liquid, having a bottom side disposed in gravitational direction and a top side disposed opposite to the gravitational direction, where the carrier liquid can be brought into contact with a granular adsorbent for the proteins, which is located in a vertically disposed first subreactor, by means of a flow of the carrier liquid around the adsorbent, so that the granular adsorbent takes up and temporarily stores proteins, and the granular adsorbent, saturated partially or completely with proteins, can be transferred into a second subreactor by means of a first pumping device or a first valve disposed on the top side and between the first subreactor and the second subreactor, so that deposition of the proteins from the granular adsorbent into a washing liquid is enabled in the second subreactor, wherein the carrier liquid can be introduced into the first subreactor by means of a first feed at a subregion, facing the bottom side, of the first subreactor, so that the granular adsorbent and the carrier liquid can flow through the first subreactor in co-current opposite to the gravitation, from the bottom side to the top side, by the carrier liquid entraining some or all of the granular adsorbent with it.

13. The apparatus according to claim 12, wherein first subreactor and/or the second subreactor are or is a fluidized bed reactor.

14. The apparatus according to claim 12, wherein washing liquid can be introduced into the second subreactor by means of a second feed on the bottom side of the second subreactor, so that the saturated granular adsorbent and the washing liquid can flow through the second subreactor in counter-current, by the washing liquid flowing from the bottom side to the top side and the granular adsorbent flowing with the gravitation from the top side to the bottom side, and in particular a washed granular adsorbent freed wholly or partially from the proteins can be transferred into the first subreactor on the bottom side of the second subreactor by means of a second pumping device or a second valve, so that repeated utilization of the granular adsorbent is enabled.

15. The apparatus according to claim 12, wherein the second subreactor has separate reaction spaces for washing, for eluting and/or for equilibrating, where in particular the respective reaction space is connected to the respectively other reaction space or respectively other reaction spaces by means of a respective transition cross section narrowed relative to a cross section of the respective reaction space.

16. The apparatus according to claim 12, wherein the second subreactor and/or the respective reaction space have or has a baffle element or a plurality of baffle elements, where the respective baffle element limits the respective cross section of the second subreactor and/or of the respective reaction space, so that a contact frequency and/or a residence time of the granular adsorbent in the second subreactor and/or in the respective reaction space is increased.

17. The apparatus according to claim 16, wherein the respective baffle element is an impact face, where the respective impact face is disposed at an angle between 60° and 80°, obliquely to a flow direction of the washing liquid.

18. The apparatus according to claim 16, wherein the respective baffle element and/or the respective impact face have or has a passage on a side facing the bottom side, so that the adsorbent can be conveyed from the top side to the bottom side by means of gravitation.

19. The apparatus according to claim 14, wherein the first pumping device and/or the second pumping device have or has a peristaltic pumping device for transferring the adsorbent from the first subreactor into the second subreactor and/or from the second subreactor into the first subreactor.

20. A method for removing proteins taken up in a carrier liquid, with the apparatus according to claim 12, comprising the steps of:
introducing the carrier liquid and the granular adsorbent on the bottom side of the first subreactor by means of the first feed or introducing the carrier liquid on the bottom side of the first subreactor by means of the first feed, said first subreactor being pre-loaded with the granular adsorbent,
bringing the carrier liquid from the bottom side of the first subreactor to the top side of the first subreactor, so that the granular adsorbent is brought in co-current with the carrier liquid and the granular adsorbent is held at least partially in suspension, so that a contact time of the granular adsorbent with the carrier liquid is increased, and
transferring the granular adsorbent from the first subreactor into the second subreactor by means of the first pumping device or the first valve, so that deposition of the proteins from the granular adsorbent into the washing liquid is enabled,
so that the proteins are removed from the carrier liquid.

21. The method according to claim 20, wherein the granular adsorbent, after the transferring into the second subreactor, is passed in counter-current with the washing liquid, so that the granular adsorbent flows with the gravitation and the washing liquid flows opposite to the gravitation.

22. The method according to claim 20, wherein the liquid is an unclarified feedstock.
